(19) United States
(12) Patent Application Publication
(10) Pub. No.: US 2012/0142087 A1
(43) Pub. Date: Jun. 7, 2012

(54) REACTOR FOR THE ANAEROBIC DIGESTION OF BIOMASS

(75) Inventor: Philippe Peultier, Valenciennes (FR)

(73) Assignee: ENERBIOM, Valenciennes (FR)

(21) Appl. No.: 13/382,006

(22) PCT Filed: Jul. 2, 2010
(86) PCT No.: PCT/FR2010/000485

§ 371 Date: Feb. 23, 2012

(30) Foreign Application Priority Data
Jul. 2, 2009 (FR) 09/03249

(51) Int. Cl. C12M 1/00

(52) U.S. Cl. 435/289.1

(57) ABSTRACT

A reactor for the anaerobic digestion of biomass, includes: a gas-tight tank that is accessible by a sealed door designed for the intake of the biomass, the tank being designed in the form of a garage; equipment for heating the biomass provided e.g. at the walls of the tank; a biogas-tapping connection; and equipment for draining the seepage liquid. According to the invention, the lower walls (6, 7) of the tank (2) define together with the side walls (8) of the tank a bowl (C) for forming a seepage liquid barrier (9) below or at the threshold of the door. The inner walls of the tank include in particular an inclined plane constituting a ramp for a vehicle and extending in the vicinity of the threshold of the door up to a horizontal wall of the bowl (C).

The invention relates to a reactor for the anaerobic digestion of biomass.

In the field of anaerobic digestion, it is known, in particular in document EP-0.023.176 a reactor for the anaerobic digestion of biomass of which the tank has the form of a garage box allowing a vehicle to quickly load or on the contrary empty the tank with fermentable materials. To this effect, the tank has a loading opening allowing for the passage of a vehicle which can be closed by a gas-tight door.

In this type of installation, the seepage liquid can be drained at the lower wall of the tank and be pumped in order to supply spray ramps, in the top portion of the tank, in order to wet the solid fermentable materials.

It is also known in document WO-2007/097392 a reactor for anaerobic digestion in the form of a garage box provided with a door allowing for the passage of a vehicle for the loading or unloading of the tank.

This reactor is provided with a biogas pipe and a device for draining the seepage liquid (percolate) provided on the bottom or on the walls of the tank.

Performance is improved by maintaining a certain level of liquid of the seepage liquid (reserve of percolate) in the tank which can be regulated thanks to a control device associated to the device for draining. The regulation controls a valve of the device for draining according to the signal of a level sensor in order to adjust the level of percolate in the tank to a preset value. In this document, the loading door must be tight not only to gas but also to liquids in order to maintain the reserve of seepage liquid. In this document, the door of the tank can be opened only when the reserve of percolate has been entirely drained, which slows down the loading and unloading operations.

It is also known in document DE-3719564 a reactor for the anaerobic digestion of biomass. This reactor includes a gas-tight tank, a biogas pipe and an opening supplied by a worm screw in order to fill the tank with fermentable materials. A device for draining makes it possible to drain the percolate which is removed via an overflow. This overflow makes it possible to define a reserve of percolate in the liquid-tight tank.

The purpose of this invention is to propose a reactor for the anaerobic digestion of biomass of which the tank allows for the creation of a guide for seepage liquid, constructed in the form of a garage allowing for the loading of the biomass thanks to a vehicle, and of which the loading or unloading of the biomass can be carried out faster than in reactors of prior art.

Another purpose of this application is to propose such a reactor of which the maintenance can be facilitated, more particularly in the case of a failure of the device for draining the percolate.

Another purpose of the invention is to propose such a reactor of simple design.

Other purposes and advantages shall appear in the following description and which is provided only for the purposes of information.

To this effect, the invention relates to a reactor for the anaerobic digestion of biomass including:
a gas-tight tank, that is accessible by a sealed door designed for the intake of the biomass, said tank being designed in the form of a garage,
an installation for heating the biomass, for example on the walls of said tank,
a biogas-tapping connection,
an installation for draining the seepage liquid.

According to the invention, the lower walls of said tank form with the lateral walls of the tank a bowl making it possible to create a reserve of seepage liquid below or at the threshold of the door.

More particularly, according to the invention, the lower walls of the tank include:
an inclined plane, constituting a ramp for a vehicle,
a horizontal wall constituting the bottom of the bowl, the inclined plane extending in the vicinity of the threshold of the door up to the bottom wall.

Advantageously, the lower walls of the tank can have, between the door and the inclined plane, a low wall rising above the threshold of the door across the entire width of the tank up to the lateral walls of said tank, except for two passages for the wheels of a vehicle. The low wall allows for the creation of a reserve of seepage liquid above the threshold of the door when said two passages are closed off.

The horizontal wall of the bowl can be located at a vertical distance from the threshold of the door between 0.5 m and 1.5 m, such as for example 1 m.

The inclined plane can extend over a horizontal distance between 5 and 15 m such as for example 10 m.

The level of the reserve of seepage liquid of the bowl can be determined by an overflow of the installation for draining.

The reactor can have a recirculation circuit for the drained seepage liquid.

The recirculation circuit can include a pumping tank supplied with drained seepage liquid.

The pumping tank can be air-tight, with a siphon provided on the drainage pipe between said bowl and said pumping tank.

Said pumping tank can further have a biogas inlet for the emptying of the pumping tank.

Said installation for heating the biomass can be provided on the walls of said tank making it possible to directly heat the biomass, or alternatively provided on the recirculation circuit of the seepage liquid making it possible to heat the biomass par spraying using the heated recirculated seepage liquid.

The invention shall be better understood when reading the description accompanied with annexed drawings wherein:

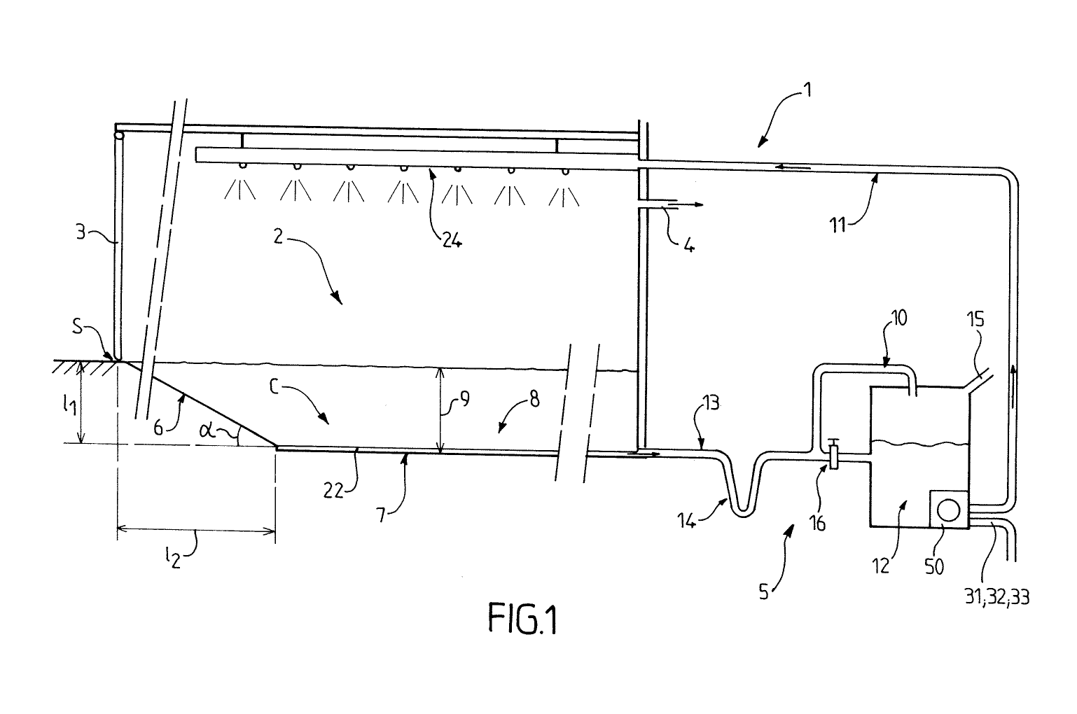

FIG. 1 is a view according to a vertical and longitudinal section view of a reactor for the anaerobic digestion of biomass in accordance with the invention according to an embodiment,

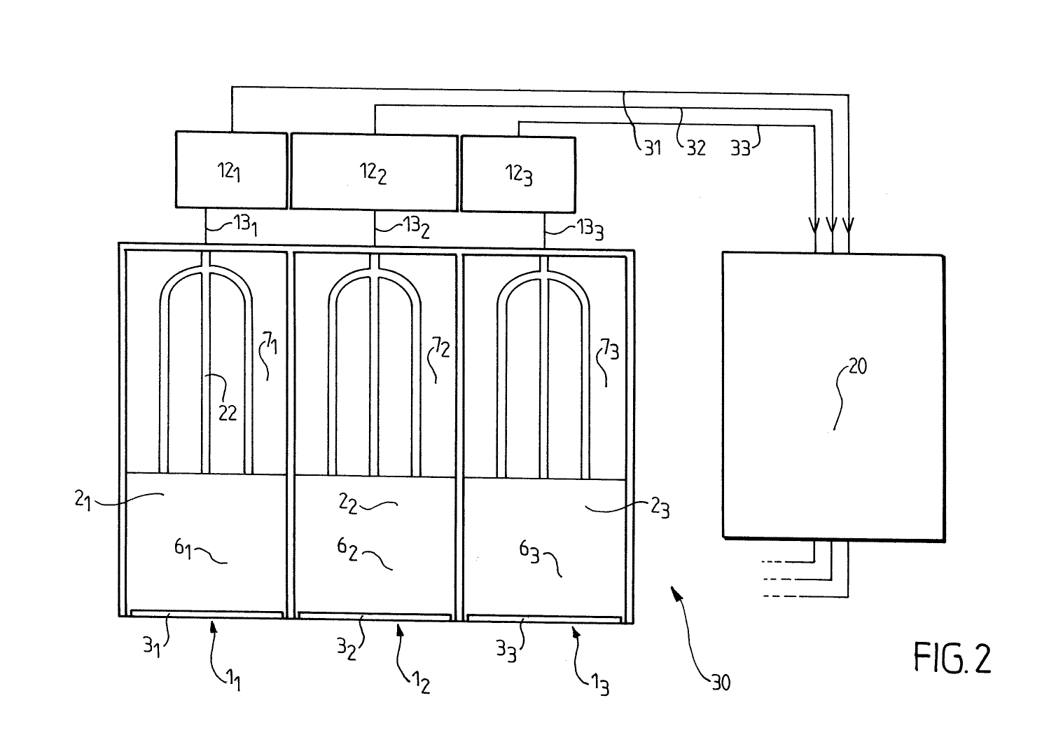

FIG. 2 is a view according to a horizontal cross-section of an installation including three reactors in accordance with the invention, in series,

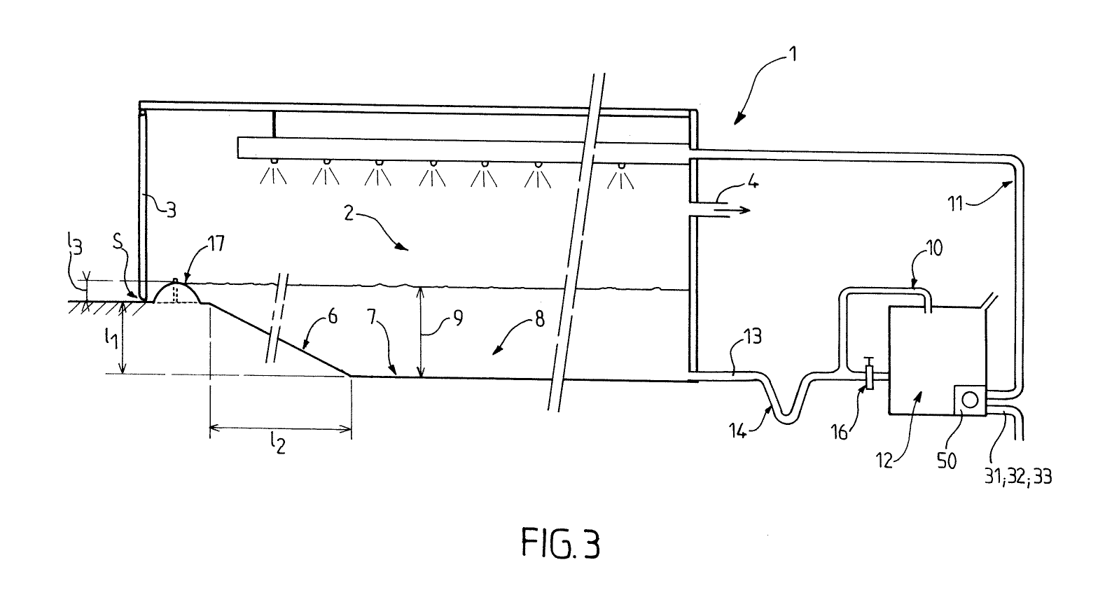

FIG. 3 is a view according to a vertical and longitudinal cross-section, of a reactor for the anaerobic digestion of biomass in accordance with the invention according to a second embodiment, having a low wall between the threshold of the door and the inclined plane,

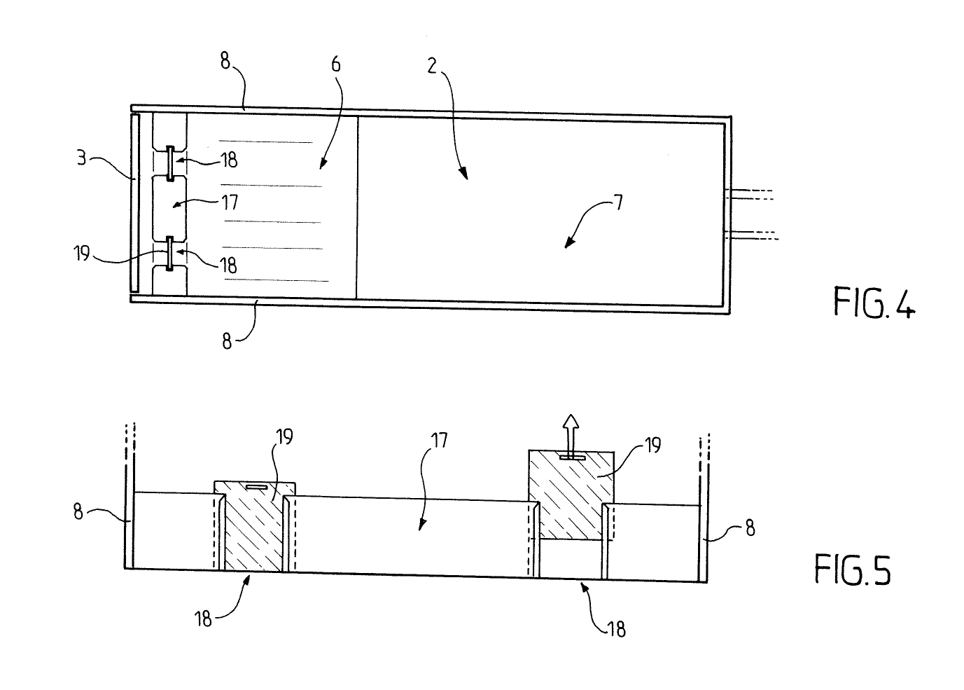

FIG. 4 is a top view of the tank of the reactor shown in FIG. 3,

FIG. 5 is a front view of the low wall of the reactor of FIGS. 3 and 4.

The invention relates to a reactor 1 for the anaerobic digestion of biomass.

This reactor includes:
a gas-tight tank 2 that is accessible by a door 3 designed for the intake of the biomass, said tank being designed in the form of a liquid-tight garage,
an installation for heating the biomass provided, for example, on the walls of said tank 2,
a biogas-tapping connection 4,
an installation for draining 5 the seepage liquid.

What is meant by garage is the fact that the tank, and more particularly the opening of the door, allow for the passage of a loading vehicle, such as a tractor, in order to load the tank with fermentable plant materials or on the contrary to unload it.

The tank can in particular be constructed of reinforced concrete.

The installation for heating can be on the walls of said tank 2 and include pipes (not shown) intended for the flow of a heat transfer fluid. These pipes can be provided on the lateral walls of the tank, in an apparent manner, or be embedded in the concrete of the lateral walls or of the lower walls of the tank.

Alternatively, the installation for heating can be provided on a recirculation circuit 11 of the drained seepage liquid, making it possible to heat the seepage liquid and as such heat the biomass by spraying the biomass using the recirculated heated seepage liquid.

According to the invention, the lower walls 6, 7 of the tank 2 form with the lateral walls 8 of said tank a bowl C making it possible to create a reserve of seepage liquid below or at the threshold S of said door.

Such as shown in FIG. 1, this bowl C is as such positioned under the threshold of the door 3, which will make it possible in particular to open the door 3 even in the case where the bowl C is filled with the seepage liquid such as in shown in FIG. 1.

Advantageously, the lower walls of the tank 2 include:
an incline plane 6, constituting a ramp for a vehicle,
a horizontal wall 7 constituting the bottom of the bowl C.

The inclined plane 6 extends as such, such as shown in FIG. 1, in the vicinity of the threshold S of the door 3 up to the horizontal wall. This ramp therefore allows a loading vehicle to descend down to the bottom of the bowl C in order to proceed with loading or unloading operations of fermentable materials.

The horizontal wall 7 constituting the bottom of the bowl is therefore located below the threshold S of the door 3, in particular at a vertical distance 1₁ of the threshold S of the door 3 between 0.5 m and 1.5 m such as for example 1 m. The reserve of seepage liquid can as such have a height in relation to the bottom between 0.5 m and 1.5 m, such as for example 1 m.

The inclined plane 6 allows the vehicle to descend starting from the threshold S of the door 3 down to the bottom of the bowl C. It can extend over a horizontal distance 1₂ between 5 m and 15 m such as for example 10 m. The slope of the inclined plane 6 allows the vehicle to descend and then rise back up without risk: this can in particular be a slope of a magnitude of 10% (between 6% and 16%) which is an angle α in FIG. 1 of a magnitude 6°,

Such as shown according to the example in FIGS. 3 to 5, a low wall 17 can be provided between the threshold S of the door 3 and the inclined plane 6. This low wall stands above the threshold S of the door 3 and extends along the entire width of the tank 2 up to the lateral walls 8 of the tank 2, except for the two passages 18 for the wheels of a vehicle.

This low wall is of a height 1₃ such as for example 40 cm, less than the ground clearance of the loading vehicle, in such a way as to be able to be crossed by the vehicle, with the wheels of the vehicle using the two passages 18, open. The separation between the two passages 18 therefore substantially corresponds to the separation between the wheels on the same axle of the vehicle.

When it is necessary to create the reserve of seepage liquid, the passages 18 can be closed off. To this effect, plates 19 are inserted in the passages 18, two opposite edges of said plate 19 cooperating with two corresponding vertical grooves of the low wall 17, provided in the passage opening 18. Once the passages 18 are closed off, the low wall allows for the creation of a reserve of seepage liquid above the threshold S of the door. This low wall makes it possible to limit the withdrawal and as such the distance 1₁ which can be 60 cm for a low wall of height 1₃ equal to 40 cm. The reserve of liquid 9 can be 1 m.

The level of the reserve of seepage liquid 9 of the bowl C can be determined by an overflow 10 of the installation for draining 5. Advantageously, the reactor 1 can include said recirculation circuit 11 of the drained seepage liquid.

This recirculation circuit 11 can include a pumping tank 12 supplied with the drained seepage liquid. The network is provided with a pump 50. Advantageously, this pumping tank 12 is air-tight so that an anaerobic reaction is maintained inside said tank 12. To this effect, a siphon 14 can be provided on the drainage pipe 13 between the bowl C and said pumping tank 12, in order to prohibit the intake of air in the pumping tank 12 from the tank 2. Furthermore, the pumping tank 12 can be provided with a biogas inlet 15 allowing for the emptying of the pumping tank 12.

Possibly, the installation for heating provided on the recirculation circuit can be provided internally to the pumping tank 12, comprising, for example, pipes, internal to the tank 12 provided for the flow of a heat transfer fluid.

We shall now describe in more detail the example embodiment shown in FIGS. 1 and 2. The installation of FIG. 2 includes several reactors 1₁, 1₂, 1₃ of which the tanks 2₁, 2₂, 2₃ and their associated doors 3₁, 3₂, 3₃ are arranged in series. The tanks 2₁, 2₂, 2₃ are built in the form of a garage made of reinforced concrete.

The drainage pipes 13₁, 13₂, 13₃ carry respectively the respective seepage liquid from said reactors 1₁, 1₂, 1₃ toward pumping tanks 12₁, 12₂, 12₃ proper to each of the reactors. The reactors 1₁, 1₂, 1₃ are constructed identically to the reactor of FIG. 1.

Pipes 31, 32, 33 make it possible to carry the liquid of each of the tanks 12₁, 12₂, 12₃ toward a storage tank 20, common.

According to the vertical cross-section view in FIG. 1, the tank 2 comprises a door 3 in the form of an articulated plate, in particular according to a horizontal axis of rotation, at its upper end at the upper wall of the tank or of the tipping type. This door can be opened or closed, actuated using a motorisation or not.

The opening of the door allows for the passage of a loading vehicle such as a tractor. The lower walls of the tank 6, 7 consist substantially of a ramp 6 and a horizontal wall 7. The inclined plane 6 extends in the vicinity of the threshold S of the door 3 up to the horizontal wall 7.

According to this example, the horizontal wall 7 is located below the threshold S of the door 3 at a vertical distance 1₁ equal to 1 m, thus allowing for the creation of a reserve of seepage liquid 9 of 1 m in height.

The inclined plane 6 extends over a horizontal distance 1₂ of 10 m. The slope is therefore 10% which corresponds to an angle α of approximately 6°. In the bowl C, the level of the reserve of seepage liquid 9 of the bowl C is determined by an overflow 10 of the installation for draining 5, of a level slightly lower than the threshold S of the door 3. Such as shown a recirculation circuit 11 of the seepage liquid is coupled to the installation for draining 5 in order o recycle the seepage liquid on the spraying ramps 24 of the seepage liquid 1 in the tank 2.

This recirculation circuit 11 includes a pumping tank 12 supplied with seepage liquid drained by the intermediary of the overflow 10. Alternatively, when it is necessary to empty the tank 2, a valve 16, normally closed, will be opened so that the liquid drained is directed by gravity in the tank 12, without passing through the overflow 10.

The tank 12 is advantageously air-tight in order to maintain the anaerobic fermentation of the seepage liquid, contained in said tank 12. In order to prohibit any intake of air in the tank 12, the drainage pipe 13 between the bowl C and the pumping tank 12 advantageously includes a siphon 14. Furthermore, the pumping tank 12 has a biogas inlet 15 for the emptying of said pumping tank 12. Also, when the pumping tank is emptied, the volume occupied by the seepage liquid will be replaced with the biogas, not air.

In order to facilitate the draining of the seepage liquid, channels 22 can be provided in depth on the horizontal wall 7 of the bottom of the bowl. The channels 22 extend over the length of the wall 7 up to the drainage pipe 13.

As such, this is a reactor for the anaerobic digestion of biomass of which the tank can be loaded with fermentable materials or on the contrary unloaded by means of a vehicle and of which the tank allows for the creation of a reserve of seepage liquid making it possible to improve the speed of fermentation in said tank.

Advantageously, in this installation, it is possible to open the door 3 while the reserve of seepage liquid is not emptied, which will make it possible to facilitate maintenance, in particular in the case of a failure of the device for draining (breakdown, closing off of the drainage pipe or others). This installation also makes it possible to increase the rhythms of filling and emptying of the tank by making possible in particular to empty the reserve of liquid 9 and to open the door simultaneously, concurrently.

The reactor 1 with the example in FIGS. 3 to 5 can be distinguished from the example in FIG. 1 by the presence of the low wall 17 between the door 3 and the inclined plane 6. This low wall 17 makes it possible, once the passages 18 are closed off by the plates 19 to create a reserve of seepage liquid of a substantial height such as for example 1 m, of which the level is above the threshold S of the door 3. This low wall makes it possible to limit the withdrawal and as such the slope of the inclined plane 6. According to this example shown, the reserve of seepage liquid has a height of 1 m. The height of the low wall 17 is 40 cm (1₃), the vertical distance 1₁ separating the threshold S of the 3 and the horizontal wall 7 is 60 cm. The horizontal distance 1₂ of the inclined plane is 10 m, the slope is 6%. The overflow 10 makes it possible to create a reserve of seepage liquid 9 above the threshold S of the door, lower than or equal to the low wall 17.

Naturally, other purposes and advantages of this invention could have been considered by those skilled in the art without however leaving the scope of the invention defined by the claims hereinafter.

1. Reactor (1) for the anaerobic digestion of biomass including:
a gas-tight tank (2) that is accessible by a sealed door (3) designed for the intake of the biomass, said tank being designed in the form of a liquid-tight garage for vehicles,
an installation for heating the biomass,
a biogas-tapping connection (4),
an installation for draining (5) the seepage liquid, characterised in that the lower walls (6, 7) of said tank (2) form with the lateral walls (8) of said tank a bowl (C) making it possible to create a reserve of seepage liquid (9) below or at the level of the threshold (S) of the door (3), said lower walls of the tank (2) including:
an inclined plane (6), comprising a ramp for a loading vehicle,
a horizontal wall (7) comprising the bottom of the bowl (C), and wherein said inclined plane (6) extends in the vicinity of the threshold (S) of the door (3) to the bottom wall (7).

2. Reactor according to claim 1 of which the lower walls of the tank (2) have between the door (3) and the inclined plane (6) a low wall (17) rising above the threshold (S) of the door over the entire width of the tank (2) up to the lateral walls (8) of the tank, except for two passages (18) for the wheels of a vehicle, with the low wall allowing for the creation of a reserve of seepage liquid (9) above the threshold (S) of the door (3) when said two passages (18) are closed off.

3. Reactor according to claim 1, wherein the horizontal wall (7) is located at a vertical distance (1₁) of the threshold (S) of the door (3) between 0.5 m and 1.5 m, such as for example 1 m.

4. Reactor according to claim 1, wherein the inclined plane (6) extends over a horizontal distance (1₂) between 5 m and 15 m, such as for example 10 m.

5. Reactor according to claim 1, wherein the level of the reserve of seepage liquid (9) of the bowl (C) is determined by an overflow (10) of the installation for draining (5).

6. Reactor according to claim 1, having a recirculation circuit (11) of the drained seepage liquid.

7. Reactor according to claim 6, wherein said recirculation circuit (11) includes a pumping tank (12) supplied with drained seepage liquid.

8. Reactor according to claim 7, wherein the pumping tank (12) is air-tight, a siphon (14) being provided on a drainage pipe (13) between said bowl (C) and said pumping tank (12).

9. Reactor according to claim 8, wherein the pumping tank (12) has a biogas inlet (15) for the emptying of said pumping tank (12).

10. Reactor according to claim 1, wherein said installation for heating the biomass is provided on the walls of said tank (2).

11. Reactor according to claim 6, wherein said installation for heating the biomass is provided on the recirculation circuit (11) of the drained seepage liquid making it possible to heat the biomass by spraying using the recirculated heated seepage liquid.

12. Installation (30) comprising several reactors (1₁, 1₂, 1₃) according to claim 1, of which said tanks (2₁, 2₂, 2₃) are arranged successively next to each other.

13. Reactor according to claim 2, wherein the horizontal wall (7) is located at a vertical distance (1₁) of the threshold (S) of the door (3) between 0.5 m and 1.5 m, such as for example 1 m.

14. Reactor according to claim 2, wherein the inclined plane (6) extends over a horizontal distance (1₂) between 5 m and 15 m, such as for example 10 m.

15. Reactor according to claim 2, wherein the level of the reserve of seepage liquid (9) of the bowl (C) is determined by an overflow (10) of the installation for draining (5).

16. Reactor according to claim 2, having a recirculation circuit (11) of the drained seepage liquid.
